(19) United States
(12) Patent Application Publication
(10) Pub. No.: US 2013/0197553 A1
(43) Pub. Date: Aug. 1, 2013

(54) CUTTING DEVICE FOR CUTTING TISSUE

(71) Applicants: Siew Weng Ng, Singapore (SG); Yau Wong, Singapore (SG)

(72) Inventors: Siew Weng Ng, Singapore (SG); Yau Wong, Singapore (SG)

(21) Appl. No.: 13/638,727

(22) PCT Filed: Mar. 4, 2011
(86) PCT No.: PCT/SG2011/000084

§ 371 Date: Nov. 26, 2012

Related U.S. Application Data
(60) Provisional application No. 61/318,803, filed on Mar. 30, 2010.

(51) Int. Cl. A61B 17/32

(52) U.S. Cl. CPC A61B 17/320016
USPC 606/170

(57) ABSTRACT

The present invention refers to a cutting device for cutting tissue or a tissue layer. A cutting device can comprise a sleeve having a first end and a second end. The sleeve may comprise a slit extending from a position at the first end of the sleeve. The first end of the sleeve may be shaped to allow insertion of the sleeve between two tissue layers. Further comprised may be a cutting rod carrying a cutting blade at a first end, the cutting rod being insertable into the sleeve through the second end of the sleeve and being movable within the sleeve. The cutting rod may be positioned within the sleeve so that the cutting blade can protrude through the slit of the sleeve. The cutting device may further comprise a support rod, the support rod being insertable into the sleeve.

### CROSS-REFERENCE TO RELATED APPLICATION

This application claims the benefit of priority of U.S. Provisional Application No. 61/318,803, filed Mar. 30, 2010, the contents of which being hereby incorporated by reference it its entirety for all purposes.

### FIELD OF THE INVENTION

The present invention refers to the field of medical devices, in particular to the field of medical devices that can be used to cut tissues and to release trigger finger.

### BACKGROUND OF THE INVENTION

Trigger finger and trigger thumb are conditions affecting the movement of the tendons as they bend the fingers or thumb toward the palm of the hand. This movement is called flexion.

The tendons that move the fingers are held in place on the bones by a series of ligaments called pulleys 130 (see FIG. 29). These ligaments form an arch on the surface of the bone 145 that creates a sort of tunnel for the tendon 140 to run in along the bone 145. To keep the tendons 140 moving smoothly under the ligaments, the tendons 140 are wrapped in a slippery coating called tenosynovium 135. The tenosynovium 135 reduces the friction and allows the flexor tendons 140 to glide through the tunnel formed by the pulleys 130 as the hand is used to grasp objects.

Triggering is usually the result of a thickening in the tendon 140 that forms a nodule, or knob. The pulley 130 ligament may thicken as well. The constant irritation from the tendon 140 repeatedly sliding through the pulley 130 causes the tendon 140 to swell in this area and create the nodule. Rheumatoid arthritis, partial tendon lacerations, repeated trauma from pistol-gripped power tools, or long hours grasping a steering wheel can cause triggering. Infection or damage to the synovium causes a rounded swelling (the nodule) to form in the tendon. Triggering can also be caused by a congenital defect that forms a nodule in the tendon. The condition is not usually noticeable until infants begin to use their hands.

The symptoms of trigger finger or thumb include pain and a funny clicking sensation when the finger or thumb is bent. Pain usually occurs when the finger or thumb is bent and straightened. Tenderness usually occurs over the area of the nodule at the bottom of the finger or thumb. The clicking sensation occurs when the nodule moves through the tunnel formed by the pulley ligaments. With the finger straight, the nodule is at the far edge of the surrounding ligament. When the finger is flexed, the nodule passes under the ligament and causes the clicking sensation. If the nodule becomes too large it may pass under the ligament, but becomes stuck at the near edge. The nodule cannot move back through the tunnel, and the finger is locked in the flexed trigger position.

The usual solution for treating a trigger digit is percutaneous release or surgery to open the pulley that is obstructing the nodule and keeping the tendon from sliding smoothly. In the case of surgery, a relatively large incision is made and the longitudinal release of the tendon sheath is performed using a scalpel. Due to the large incision needed, this may increase the risk of infection and thus prolong the recovery. As for the percutaneous release, a needle is used to release the locked finger.

Unfortunately, there is a high risk of damaging the digital artery, nerve and also slitting of the underlying tendon due to the direction of insertion. A non surgical method would include the injection of a steroid hormone, such as cortisone, into the tendon sheath. This can decrease the inflammation and shrink the nodule to relieve the triggering, but the results will probably be short lived.

Thus, it is an object of the present invention to develop further methods and devices which can be used in the treatment of trigger fingers and which overcome at least some of the problems referred to above.

### SUMMARY OF THE INVENTION

In one embodiment, the present invention refers to a cutting device for cutting tissue. This cutting device comprises a sleeve having a first end and a second end. The sleeve may comprise a slit extending from a position at the first end of the sleeve. The first end of the sleeve may be shaped to allow insertion of the sleeve between two tissue layers. Further comprised may be a cutting rod carrying a cutting blade at a first end, the cutting rod being insertable into the sleeve through the second end of the sleeve and being movable within the sleeve. The cutting rod may be positioned within the sleeve so that the cutting blade can protrude through the slit of the sleeve. The cutting device may further comprise a support rod, the support rod being insertable into the sleeve through the second end of the sleeve and being movable within the sleeve. The cutting rod and the support rod may fit together into the sleeve. When the cutting rod and the support rod are arranged together within the sleeve the cutting blade of the cutting rod is protruding through the slit of the sleeve.

In a further embodiment, the cutting rod and the support rod may be movable independently of each other within the sleeve and insertable in the sleeve.

In a further embodiment, the cutting rod and the support rod may fill out substantially the entire space of the sleeve when fully inserted into the sleeve during use.

In a further embodiment, the shape of the cutting rod and the shape of the support rod can be the same or different from each other.

In one embodiment, the sleeve may be round and the cutting rod and the support rod may have a semicircular shape.

In another embodiment, the first end of the sleeve comprises a tip with a tapered shape. For example, in one embodiment, the first end of the sleeve may comprise a tip beveled along a vertical axis of the first end.

In another embodiment, the cutting device of the present invention may further comprise a housing comprising a first end and a second end. The housing can be connected at the first end to the sleeve. The housing may comprise a movable cutter holder and a movable support rod holder. The cutter holder may support the cutting rod and the support rod holder supports the support rod. The cutter holder and the support rod holder may be movable independently from each other. During use, a movement of the cutter holder within the housing allows moving the cutter rod within the sleeve and a movement of the support rod holder allows moving the support rod within and out of the sleeve.

In another embodiment, the first end of the first housing may be closed with a front cap which is connected to the sleeve and the second end may be closed with an end cap.

In another embodiment, the cutter holder and the support rod holder may be movably supported by a guiding member. The guiding member can be comprised within the housing.

In still another embodiment, the cutting device further may comprise a tensible member. The tensible member may be positioned between the first end of the first housing and the cutter holder such that a movement of the cutter holder towards the first end of the first housing cocks the tensible member and positions the cutter holder in a cocked position.

In another embodiment, the cutting device can further comprise at least one side guide mounted at a side of the housing. The side guide can comprise an engaging region located near the first end of the housing. A protrusion located at at least one side of the cutter holder engages the engaging region of the at least one side guide through an opening of the housing to keep the cutter holder in the cocked position when the tensible member is cocked.

In another embodiment, the cutting device comprises two side guides wherein the two side guides are positioned at opposing sides of the housing.

In another embodiment, the cutter holder further comprises at least one slit separating the cutter holder into a central core region and a side member. The central core region may support the cutting rod and may provide a contact region for the tensible member.

The side member may comprise the protrusion which engages the engaging region of the at least one side guide to keep the cutter holder in the cocked position when the tensible member is cocked. The side member may be movable towards the central core region from which it is separated by the slit upon exerting pressure on the side member; wherein a movement of the side member of the cutter holder towards the central core region allows to uncock the cutter holder from its cocked position by disengaging the connection between the protrusion and the side guide. Uncocking the tensible member and thus the cutter holder results in the cutter holder to move towards the end cap of the cutting device. During this backwards movement the cutting rod with the raised cutting blade moves backwards within the sleeve. During this movement, the protruded cutting blade cuts the overlying tissue layer.

In another embodiment, the cutter holder may comprise two slits separating a section of the cutter holder into a central core region and two side members which are located at opposing sides of the cutter holder.

In still another embodiment, the second end of the housing may comprise a first opening for inserting a control rod into the device. The first opening may be positioned to allow the control rod to engage the cutter holder and to move the cutter holder towards the first end of the housing when inserted into the device.

In another embodiment, the second end comprises a second opening for inserting a retrieval rod into the cutting device or for inserting a control rod into the cutting device. The second opening may be positioned to allow a retrieval rod to engage the support rod holder and to move the support rod holder towards the second end of the housing when inserted into the device and thus moving the support rod supported by the support rod holder out of the sleeve. Furthermore, the second opening may be positioned to allow the control rod to move the support rod holder towards the first end of the housing and thus moving the support rod supported by the support rod holder into the sleeve.

In another embodiment, the cutting device may further comprise a support rod guiding member connected to the support rod and comprising a through hole extending from one end of the support rod guiding member to the other end.

In still another embodiment, the cutting rod may be adapted to fit through the through hole of the support rod guiding member.

In another embodiment, the sleeve may be connected to a positioning member. The positioning member allows positioning of the sleeve at a specific position in a tissue of a subject.

In another aspect, the present invention is directed to a control rod for use in a cutting device of the present invention. The control rod may comprise a first end and a second end and comprises a first section and a second section. The first and the second section may have different diameters. The diameter of the first section located at the first end of the rod may be smaller than the diameter of the second section following the first section.

In another embodiment, the control rod further comprises a handle located at the second end of the control rod.

In still another embodiment, the diameter of the first section of the control rod may be adapted to be insertable through the first opening of the second end of the housing of the cutting device and the diameter of the second section may be adapted to be larger than the diameter of the first opening of the second end of the housing of the cutting device.

In another embodiment, the diameter of the second section of the control rod may be adapted to be insertable through the second opening of the second end of the housing of the cutting device.

In another aspect, the present invention is directed at a retrieval rod for use in a device of the present invention. The retrieval rod may comprise a first end and a second end. The retrieval rod may comprise an engaging member at the first end of the retrieval rod for engaging the support rod holder.

In another embodiment, the retrieval rod may further comprise a handle located at the second end of the control rod.

In another embodiment, the engaging member of the retrieval rod may comprise a thread.

In another aspect, the present invention is directed to a support rod guiding member for use in a cutting device of the present invention. The support rod may comprise a through hole extending from one end of the support rod guiding member to the other end.

In another aspect, the present invention is directed to a system for cutting tissue. The system may comprise a cutting device of the present invention, a control rod of the present invention, and a retrieval rod of the present invention.

In another embodiment the system may further comprise an ultrasound device.

### BRIEF DESCRIPTION OF THE DRAWINGS

The invention will be better understood with reference to the detailed description when considered in conjunction with the non-limiting examples and the accompanying drawings, in which:

FIG. 1 shows a schematic view of an embodiment of cutting device.

FIG. 2 shows an enlarged view of a sleeve 3100 and a positioning member 3004 of an embodiment of a cutting device. The inset in FIG. 2 shows an enlarge view of the first end of the sleeve 3100.

FIG. 3 shows different embodiments of tips 1, 3002 connectable to a sleeve.

FIG. 4 shows a schematic view of a support rod guiding member.

Fig, 5 shows a schematic view of a cutting rod.

FIG. 6 illustrates different shapes which can be used for a cutting rod 2 and a support rod 6.

FIG. 7 shows a perspective view of an embodiment of a cutting device.

FIG. 8 shows an exploded perspective view of a cutting device from two different angles.

FIG. 9 shows a schematic view of a support rod holder 7, and a cutter holder 8 from the bottom.

FIG. 10 shows a schematic view of an assembly of support rod holder 7, a cutter holder 8, and guiding member 9.

FIG. 11 shows a schematic side view of a cutting device without a side guide 12 and a tensible member 5.

FIG. 12 shows a photograph and a schematic side view of a cutting device without the side guide 12 but including the tensible member 5.

FIG. 13 shows a photograph and a schematic oblique view of a cutting device.

FIG. 14 shows a schematic view of a cutting rod 2.

FIG. 15 shows a schematic view of a support rod 6.

FIG. 16 illustrates a schematic view of a support rod holder 7 showing the front side and back side of a support rod holder 7.

FIG. 17 shows a schematic top view of a support rod holder 7 and support rod 6.

FIG. 18 shows a schematic front view and back view of a front cap 11 of a housing 10.

FIG. 19 shows a perspective view of a sleeve 3.

FIG. 20 shows a schematic view of a tangible member, namely a spring 5.

FIG. 21 shows a schematic view of an end cap 4 of a housing 10.

FIG. 22 shows different perspective views of a cutter holder 8.

FIG. 23 shows a schematic view, of a guiding member 9.

FIG. 24 shows a schematic view of a housing 10.

FIG. 25 shows a schematic view of a side guide 12.

FIG. 26 shows a schematic view of a retrieval rod 500.

FIG. 27 shows a schematic view of a control rod 550.

FIG. 28 illustrates the different sequences during the use of the cutting device described herein. The dotted line labelled with “A” indicates the position for the cross-sectional illustration (Section A-A) of the cutting device shown under the top view of the cutting device.

### DETAILED DESCRIPTION OF THE PRESENT INVENTION

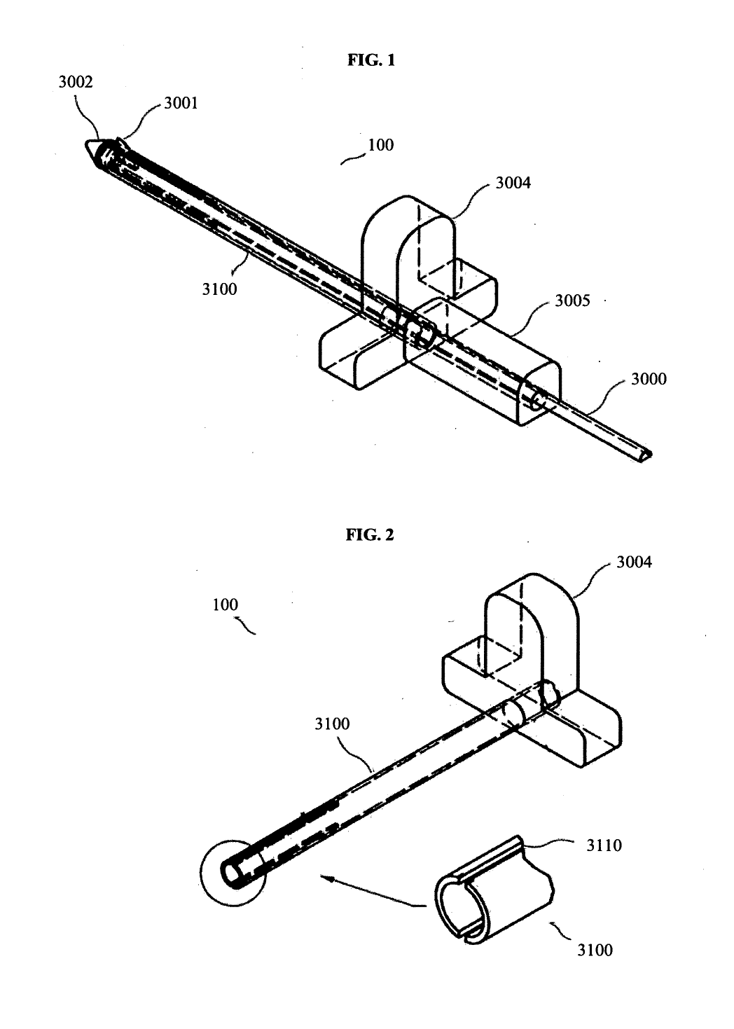

In a first aspect the present invention refers to a cutting device 100 for cutting tissue. In one embodiment, the cutting device 100 is for releasing, a trigger finger. Examples of different embodiments of cutting devices are shown for example in FIGS. 1, 7 and 8. In general, a cutting device 100 of the present invention comprises a sleeve 3, 3100 having a first end and a second end. The term sleeve refers in general to a tube like structure, i.e. a hollow axle. A sleeve is designed to fit over another part or parts. The sleeve 3, 3100 may comprise a slit 31, 3110 extending from a position at the first end of the sleeve 3, 3100. The first end of the sleeve 3, 3100 may be shaped to allow insertion of the sleeve between two tissue layers.

Further comprised may be a cutting rod 2, 3000 carrying a cutting blade 13, 3001 at a first end, the cutting rod 2, 3000 being insertable into the sleeve 3, 3100 through the second end of the sleeve 3, 3100 and being movable within the sleeve 3, 3100. The cutting rod 2, 3000 may be positioned within the sleeve 3, 3100 so that the cutting blade 13, 3001 can protrude through the slit 31, 3110 of the sleeve 3, 3100. The cutting device may further comprise a support rod 6, 3201, the support rod 6, 3201 being insertable into the sleeve 3, 3100 through the second end of the sleeve 3, 3100 and being movable within the sleeve 3, 3100. The cutting rod 2, 3000 and the support rod 6, 3201 may fit together into the sleeve 3, 3100. When the cutting rod 2, 3000 and the support rod 6, 3201 are arranged together within the sleeve 3, 3100 the cutting blade 13, 3001 of the cutting rod 2, 3000 is protruding through the slit 31, 3110 of the sleeve 3, 3100.

To use the above cutting device, a small transverse skin incision is made in the proximity of the affected area in which it is intended to cut the pulley 130. Prior to the insertion of the first end of the sleeve 3, 3100 of the cutting device 100 into the incision, the cutter rod 2, 3000 should be in the fully inserted position within the sleeve 3, 3100. As long as the support rod 6, 3201 is not fully inserted into the sleeve the cutting blade 13, 3001 will not protrude through the slit 31, 3110 of the sleeve 3, 3100.

Under the guidance of an ultrasound machine, the sleeve 3, 3100, with the cutter rod 2, 3000 fully inserted, is inserted between the pulley 130 and the tendon 140 (see FIG. 29). In doing so, the pulley 130 will be stretched taut. This will facilitate the slitting of the pulley 130 thus releasing the trigger finger. When in position, the support rod 6, 3201 is inserted fully into the sleeve 3, 3100. This will force the cutting blade 13, 3001 to protrude and pierce through the pulley 130. The cutter rod 2, 3000 is then pulled backward to longitudinally slit the pulley 130. This step may be repeated if the desired release is not achieved in the first cut. When the desired release has been achieved, the support rod 6, 3201 is withdrawn to allow the blade 13, 3001 on the cutter rod 2, 3000 to be retracted and the cutting device 100 is withdrawn as a whole. This will prevent any unnecessary damage to the surrounding tissues, such as the digital artery, a nerve and the underlying tendon. The above device cannot only be used for cutting the pulley to release a trigger finger but can also be used for cutting of other tissue layers.

FIGS. 1 and 2 illustrate an embodiment of a cutting device 100 of the present invention. In this embodiment, the circular shaped sleeve 3100 of the cutting device 100 is connected to a positioning member 3004. The sleeve 3100 can be connected to the positioning member 3004 in any known manner. For example, the sleeve 3100 can be welded to the positioning member 3004 or can be removably inserted into the positioning member 3004, for example by a push-fit mechanism.

The positioning member 3004 can have any shape which allows the operating personnel to easily hold and move the connected sleeve 3100 during its insertion into the affected area. In the embodiment illustrated in FIGS. 1 and 2 the positioning member 3004 has a T-shape wherein the sleeve 3100 is connected to the horizontal bar of the T-shaped positioning member 3004. The positioning member 3004 comprises a through-hole at the point of connection between the sleeve 3100 and the positioning member 3004 which allows inserting and removing of the cutter rod 3000 and the support rod 3201.

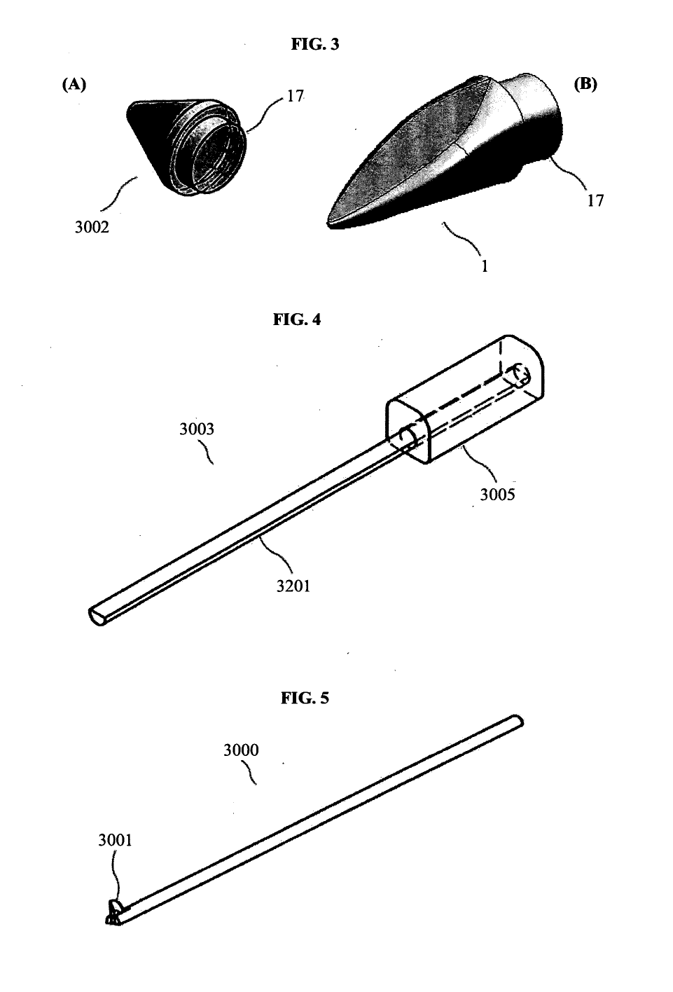

To aid the insertion of the sleeve 3, 3100 the first end of the sleeve ends can have a tapered shape. In one embodiment a tip is fixed to the first end of the sleeve. Examples of such tips are illustrated in FIG. 3. FIG. 3(A) shows a tip having the shape of a cone 3002.

FIG. 3(B) shows a tip bevelled along a vertical axis. However, the tip can also have other shapes which allow to more easily insert the sleeve at the affected are of the body.

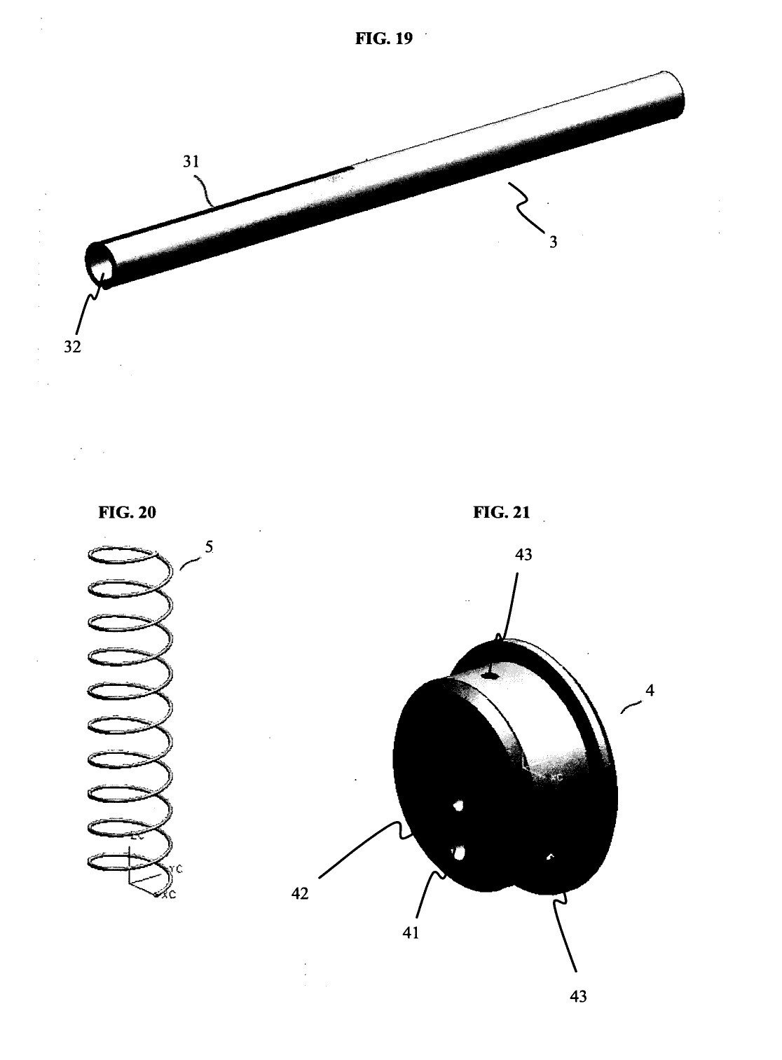

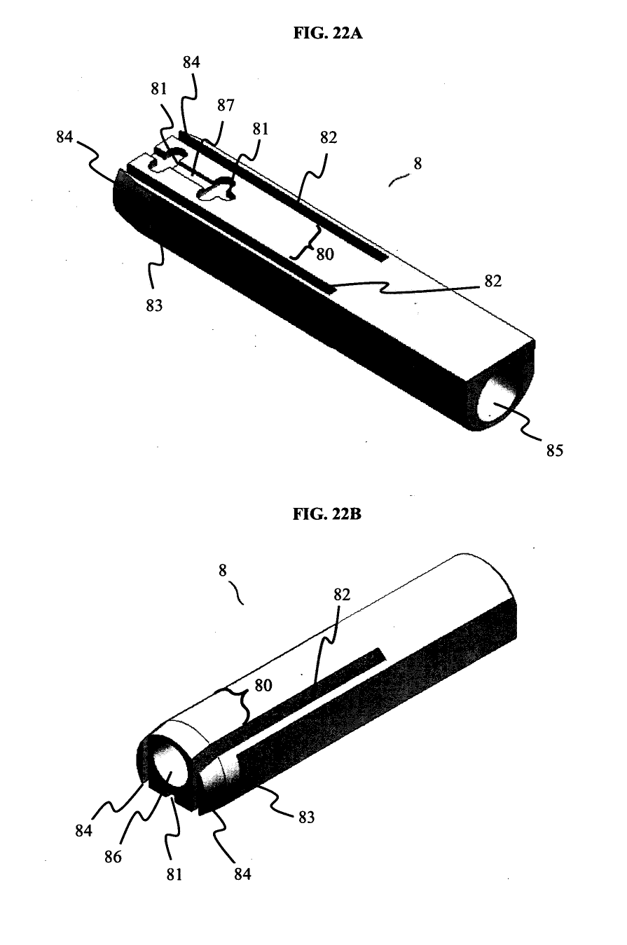

The tips shown in FIG. 3 (A) and (B) comprise a connecting collar 17. The connecting collar 17 allows attaching the tip 1, 3002 to the open end 32 of a sleeve (see e.g. inset in FIG. 2 or FIG. 19). The collar serves as a push fit fitting and is protruding from the end of the tip which is to be inserted into the sleeve opening. A pointed tip also assists to separate the different tissue layers from each other when inserting it with the pointed end of the tip ahead between the tissue layers. In the present invention, separating the different tissue layers from each other also avoids that the tissue layer which lies on the side of the sleeve through which the cutting blade does not protrude, in the present case the tendon, gets damaged during the cutting process.

FIG. 1 shows the situation in which the cutter rod 3000 carrying the cutting blade 3001 near its first end is protruding through the slit 3110 of the sleeve 3100. The cutting blade 3001 of the cutter rod 3000 only protrudes through the slit 3110 of the sleeve 3100 in case the support rod 3201 (see FIG. 4) is inserted into the sleeve 3100. The cutter rod 3000 is dimensioned to rest in a lower position within the sleeve 3100 if the support rod 3201 is not inserted into the sleeve. In the embodiment illustrated in FIG. 1 and FIG. 4, the support rod 3201 and the cutter rod 3000 have a semicircular shape. Together they form a circle which fills almost the entire space of the hollow sleeve when they are inserted together in the sleeve 3100.

In one embodiment, the cutting device 100 is inserted between the tissue layers while the support rod 3000 is not inserted in the sleeve or inserted only to such extend that the cutting blade 3001 is not protruding through the slit 3110 of the sleeve 3100. Insertion of the support rod 3201 raises the cutter rod 3000 into its upper position within the sleeve in which the cutting blade 3001 is protruding through the slit 3110 of the sleeve 3100. The cutting process of the tissue layer is carried out while the support rod 3201 is fully inserted into the sleeve or inserted into the sleeve to such extend that the cutting blade 3001 protrudes through the slit 3110 of the sleeve 3100 during the entire cutting process.

FIG. 1 illustrates the situation in which the cutter rod 3000 and the support rod 3201 (not visible in FIG. 1 because it is fully inserted in the sleeve) are inserted in the sleeve. In this situation, to cut a tissue layer through which the cutting blade 3001 cuts, the cutter rod 3000 is pulled back while the support rod 3201 remains in position within the sleeve. To bring the cutter rod 3000 and the cutting blade back into the starting position before the cut shown in FIG. 1, the support rod 3201 is removed from the sleeve 3100. Removing the support rod 3201 brings the cutter rod 3000 back into its lower position in which the cutting blade 3001 is hidden within the sleeve 3100 or in other words in which the cutting blade 3001 is not protruding through the slit 3110 of the sleeve to avoid damaging surrounding tissue. To repeat the process the support rod 3201 is re-inserted into the sleeve 3100 to bring the cutting rod 3000 back into its upper position in which the cutting blade 3001 protrudes through the slit 3110 of the sleeve 3100.

As shown in FIGS. 1 and 4, the support rod 3201 can be connected to a handle 3005 which allows the practitioner to easily insert the support rod 3201 into the sleeve 3100. The handle provides a through hole which fits the size of the cutter rod 3000. As illustrated in

FIG. 1, the through hole in the handle 3005 allows inserting the support rod 3201 into the sleeve while the cutter rod 3000 is already inserted in the sleeve 3100. The handle 3005 can have any shape which allows easy handling of the support rod guiding member 3003. In the illustration of FIG. 4, the support rod guiding member 3003 comprising the handle 3005 and the support rod 3201 has a rectangular block shape wherein the upper corners and/or the lower corners are rounded.

As shown in the inset of FIG. 2, the sleeve 3100 can comprise a double slit, i.e. two slits 3110 located on opposite sides of the sleeve 3100. However, it is also possible that the sleeve 3100 comprises only one slit 3110 as shown for example the embodiment illustrated in FIG. 13.

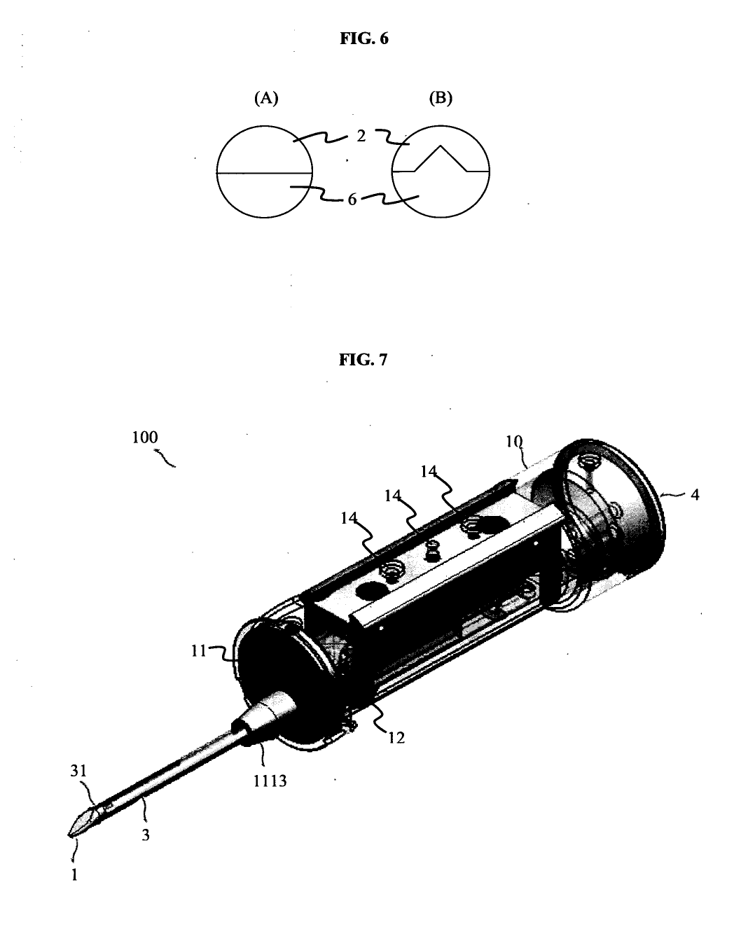

In the embodiment illustrated in FIGS. 1 to 5, the support rod 3201 and the cutter rod 3000 have a semicircular shape. However, the shape of the support rod 3201 and the cutter rod 3000 is not limited to a semicircular shape. The shape of the support rod 3201 and the cutter rod 3000 can have any shape as long as the shape is complementary to each other, i.e. the support rod 3201 and the cutter rod 3000 together form a shape which allows insertion into the sleeve and protruding of the cutting blade through the slit of the sleeve when both support rod and cutter rod are inserted in the sleeve. FIG. 6 illustrates further possible shapes for the support rod and cutter rod. FIG. 6 (A) illustrates the example also shown in FIG. 1. In FIG. 6 (B) the cutter rod 2 and the support rod 6 have an irregular shape but their shapes are complementary to each other and form a full circle. It would also be possible that in case a rectangular sleeve is used the support rod and the cutter rod form together a rectangular form or even a circular form as long as the resulting form fits into the sleeve and allows the cutting blade to protrude through the slit of the sleeve.

The slit on the sleeve is extending from a first end of the sleeve towards the second end. The length of the slit can vary. The length of the slit may be adapted to conform to the length of the cut which one intends to make using the cutting device 100 of the present invention. The length of the slit determines the distance that the cutting blade 13 on the cutting rod 2 can cover. For example, the slit can extend from the first end of the sleeve at which also the tip is connected to about 10, 20, 30, 40, 50, 60, 70, 80, 90 or 100% of the entire sleeve.

The cutting blade 13, 3001 can have any shape as long as it is suitable to pierce through a tissue or tissue layer and cut the respective tissue or tissue layer, such as the pulley 130 in case of releasing a trigger finger. In one embodiment, the blade edge of the cutting blade is pointing away from the first end of the sleeve, i.e. the end with the tapered shape or the end to which the tip 1, 3002 is mounted. Thus, the blade edge is facing the second end of the sleeve which is the end through which the cutting rod 2, 3000 and the support rod 6, 3201 are inserted into the sleeve. The blade 13, 3001 can be made of any known material which is known for the manufacture of knifes, such as ceramic materials or stainless steel. In one embodiment, the blade is obtained from the tip of a blade used for scalpels.

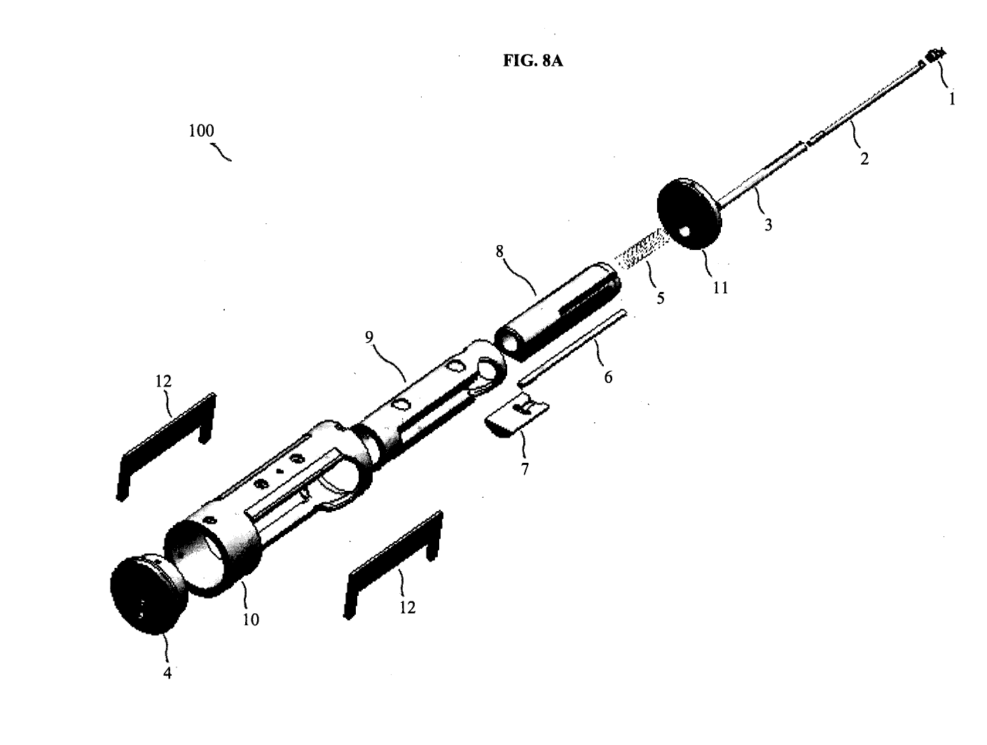

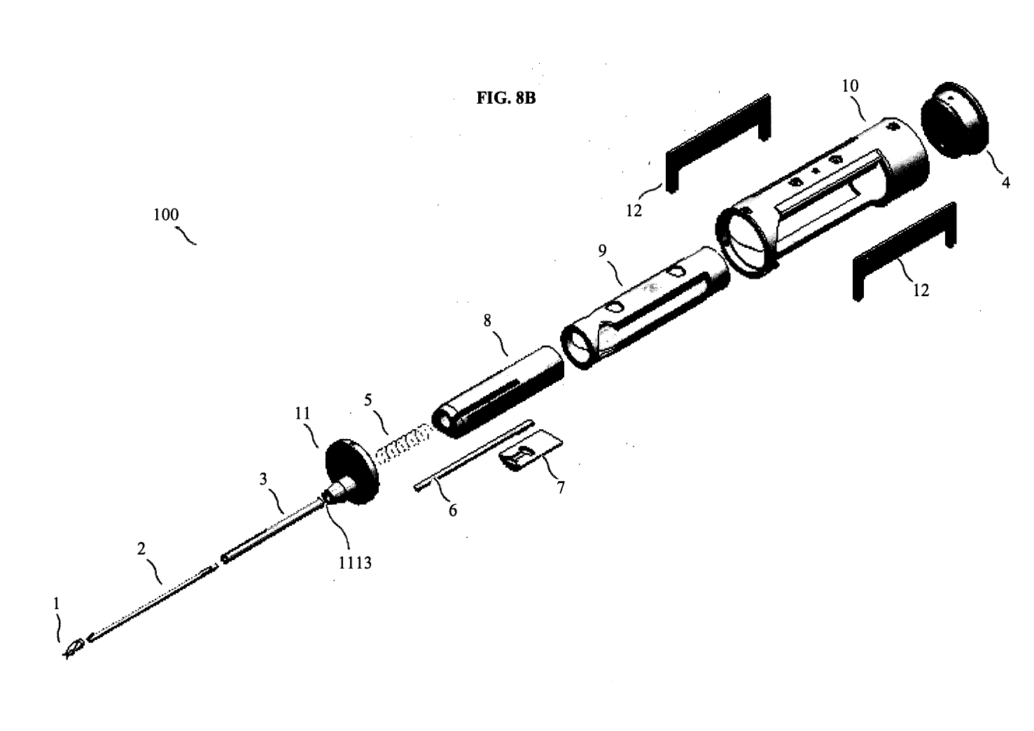

In another embodiment, the cutting device 100 may further comprise a housing 10 comprising a first end and a second end as illustrated for example in FIGS. 7 and 8. The housing 10 can be connected at the first end to the sleeve 3. The housing 10 may comprise a movable cutter holder 8 and a movable support rod holder 7. The cutter holder 8 may support the cutting rod 2, and the support rod holder 7 may support the support rod 6. The cutter holder 8 and the support rod holder 7 may be movable independently from each other. During use, a movement of the cutter holder 8 within the housing 10 allows moving the cutter rod 2 attached to it within the sleeve 3 and a movement of the support rod holder 7 allows moving the support rod 6 within and out of the sleeve 3.

While the principal working mechanism for cutting of tissue or a tissue layer is the same as described for the embodiment of the cutting device shown in FIG. 1, this further embodiment comprises a semi-automated cutting mechanism. This mechanism enables the practitioner to conduct the cutting process by simply exerting a force on the release mechanism of the cutting device 100 described herein after it has been positioned in the affected are of the body.

Therefore, the cutting device 100 illustrated in FIGS. 8 (A) and 8 (B) may comprise in addition to the tip 1, the cutter rod 2, the sleeve 3 and the support rod 6, a housing 10 comprising a front cap 11 and an end cap 4. Within the housing may be comprised a guiding member 9 which again wraps around a cutter holder 8 and a support rod holder 7. Between the cutter holder 8 and the front cap 11 a tensible member, in this case a spring 5, may be arranged. The spring 5 serves to activate the semi-automated release mechanism which upon its release draws back the cutting rod 2 through the sleeve 3 and thus the cutting blade 13 which carries out the cut of the tissue or tissue layer.

For cocking of the release mechanism the tensible member 5 arranged between the front cap 11 of the housing 10 and the cutter holder 8 is compressed. The cutting rod 2 is attached to the cutter holder 8. Compression of the tensible member 5 is achieved by pushing the cutter holder 8 towards the first end of the housing, i.e. towards the front cap 11. The forward movement of the cutter holder 8 moves the cutter blade 2 towards the first end of the sleeve 2. After compression of the tensible member 5, the cutter holder 8 may be arrested in the cocked position through side members 84 which engage the engaging region 122 of a side guide 12 via a protrusion 83 located at the front end of the cutter holder 8 facing the front cap 11. In this cocked condition the cutting device can be inserted at the affected area of the body.

However, it is also possible to cock the release mechanism for the cutter rod 2 after insertion of the cutting device 100 at the affected area of the body. Once the cutter device 100 is placed, the support rod 6 is pushed forward into the sleeve thus raising the cutting blade 13 through the slit 31 of the sleeve 3. To release the mechanism a force is applied directly to side member 84. The side member 84 moves towards a central core region 80 of the cutter holder thus disengaging the protrusion 83 which engages the engaging region 122 of the side guide 12. Upon disengaging the compressed tensible member 5 relaxes and moves the cutter holder 8 towards the second end of the housing 10, i.e. towards the end cap 4 of the housing 10. In other words, as result of the disengagement, the cutter holder 8 is pushed back by the uncocked tensible member 5 towards the second end of the housing 10. This backwards movement of the cutter holder 8 moves the attached cutting rod 2 and the cutting blade 13 protruding through the slit 31 backwards which causes the cut of the overlying tissue or tissue layer by the cutting blade 13 protruding through the slit 31.

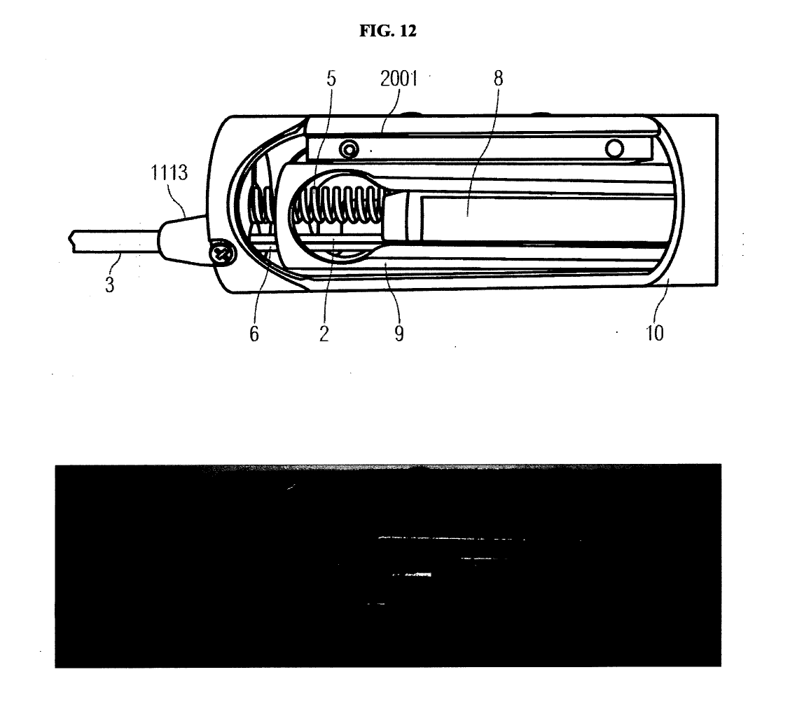

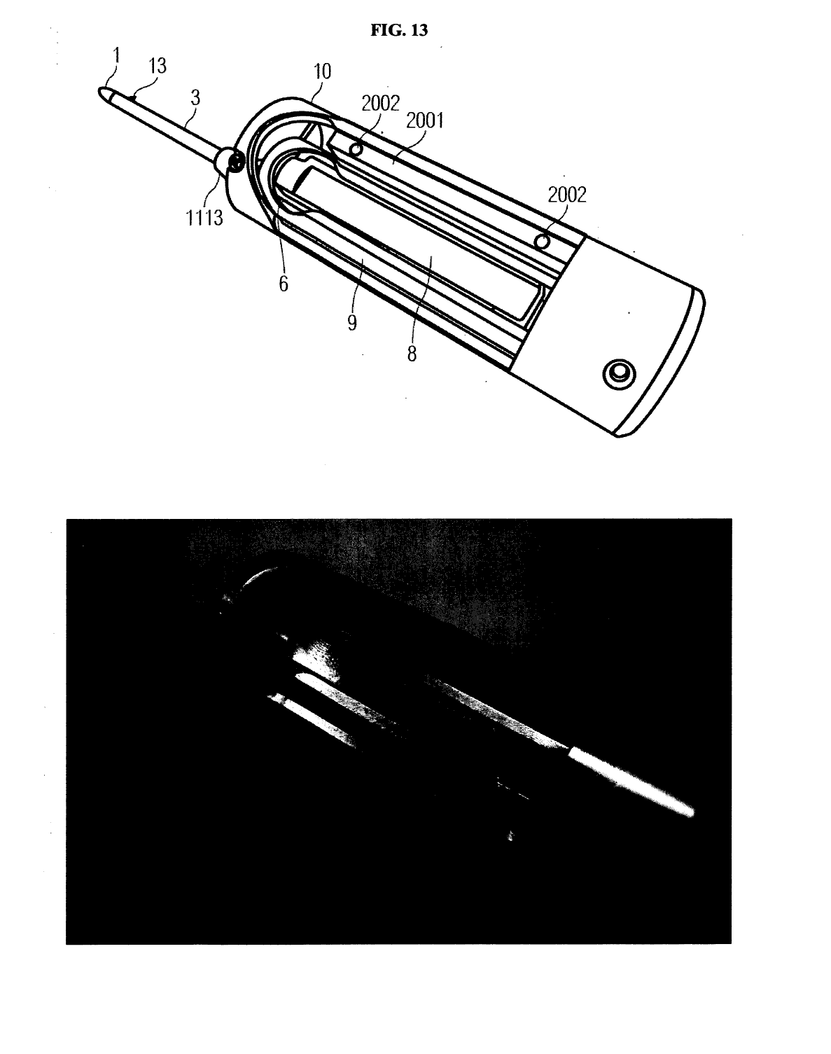

FIGS. 11 and 12 show for example details of the components which form part of the release mechanism described herein. In FIGS. 11 and 12, a front cap 11 is attached to the housing 10. The side of the front cape facing the inside of the housing comprises an opening 1110 through which the cutter rod 2 and the support rod 6 can be introducible into the sleeve 3. The cutter rod 2 is attached to the cutter holder 8 and the support rod 6 is attached to the support rod holder 7, which is located in direct proximity to the cutter holder 8 (not shown in FIGS. 11 and 12) within the guiding member 9. In FIG. 11 the guiding member 9 has been moved aside to illustrate the positioning of the cutter rod 2 and the support rod 6 within the housing 10.

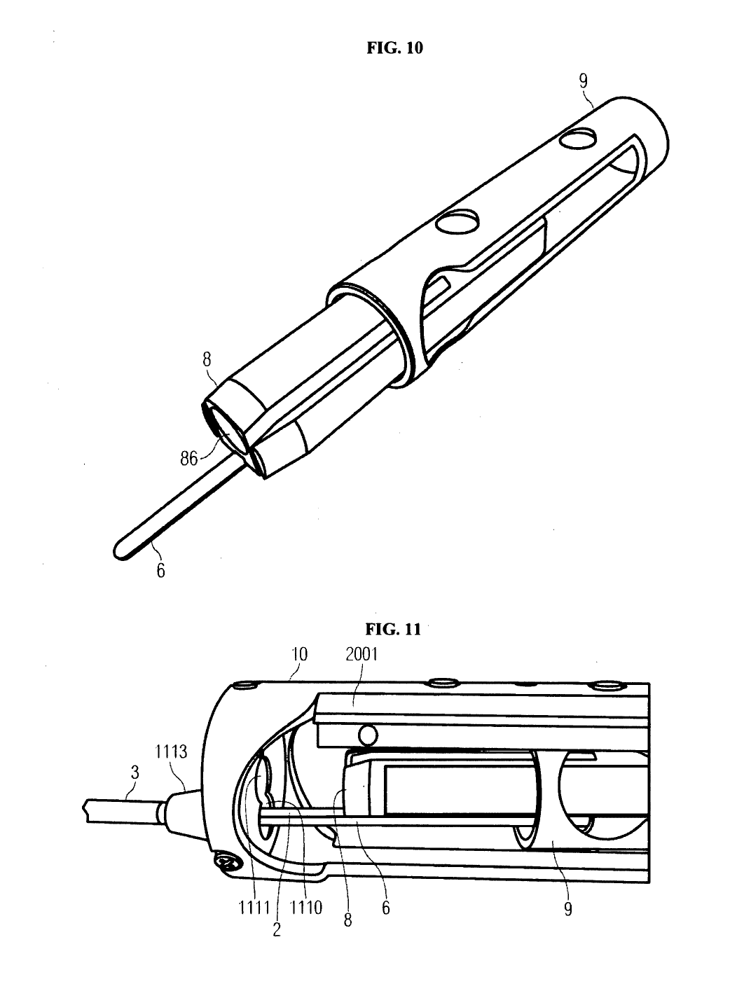

The front cap 11 of the housing further comprises a contact area for the tensible member which may be located directly above the opening 1110 for the cutter rod 2 and the support rod 6. This contact area can be a depression comprised in the front cap as shown in FIG. 11 but can also be a protrusion which functions like a flange or fitting for positioning the tensible member in its position at the front cap. 11. The tensible member 5 may be attached to the front side of the cutter holder 8 in the same manner as the tensible member 5 is attached to the front cap 11 of the housing 10. In one embodiment illustrated in FIGS. 10 and 22(B), the depression can extend into an opening or recess 86 which envelopes a section of the tensible member 5.

The opening 86 in the central core region 80 of the cutter holder 8 may not extend through the entire length of the cutter holder 8 but only to an extent sufficient for the tensible member 5 to be securely supported by the cutter holder 8 so as to avoid derailing of the tensible member 5 upon movement of the cutter holder 8 towards the front cap 11 of the housing 10. The opening 86 can have any shape as long as it sufficiently supports the tensible member 5. FIG. 12 illustrates a situation in which the uncocked tensible member is attached to the front cap via contact to the contact area 1111 of the front cap 11 on the one side and to the cutter holder 8 via insertion through the opening 86 into the cutter holder 8.

FIG. 18 illustrates close up views of the front cap 11 to be attached to the housing 10 of the cutting device 100. The front cap 11 has a shape which allows it to be fit to the housing 10 of the cutting device 100. At the side which is facing inside the housing 10, the front cap comprises an opening 1110 which extends through the front cap to the front side thereof 1114. As illustrated in FIG. 18 (A), the front side of the cap 11 comprises a connecting member 1113 in form of a protrusion with a narrowed end section for fitting in the sleeve 3. The connecting member 1113 serves as a track guide for the cutter rod 2 and the support rod 6 which can be inserted in and out of the sleeve 3 via the opening 1110 and 1114 of the front cap 11. The front cap further comprises orifices 1112 for fixation means, such as screws, rivets or pins which can be used to attach the front cap to the housing 10. The diameter of the through-hole through the front cap may narrow from a larger diameter at the opening 1110 at the back side of the front cap 11 to the opening 1114 at the front side of the front cap 11 for guiding the cutting rod 2 and the support rod 6 into the connected sleeve 3.

Besides the mounting spot for the tensible member at the cutter holder 8, FIGS. 22A and 22B show further elements which may form part of the release mechanism. The cutter holder 8 can be divided in a central core region 80 which is separated either to one side or to both sides via slits in the cutter holder from the side member(s) 84. The slit(s) 82 extends from the front end of the cutter holder 8 to a section near the end of the cutter holder 8 and separates a section of the cutter holder along a vertical axis from the bottom to the top to form a flexible side member(s) at the side of the cutter holder.

The side member(s) 84 thus formed may be thinner that the central core region 80. The side member(s) 84 has a thickness which allows it to easily move the side member(s) 84 towards the central core region 80 upon exertion of a force on the side of the side member 84 facing away from the central core region 80. Therefore, the side member 84 can also be referred to as resilient member 84.

The side member(s) 84 comprises at its front section which is pointing towards the front cap 11 of the housing a protrusion 83. The protrusion 83 is formed on the side member(s) 84 in a position which allows the protrusion 83 to engage a part of the cutting device 100 upon cocking of the tensible member 5 to arrest the cutter holder 8 in the cocked position. Upon compression of the tensible member 5 and thus movement of the cutter holder 8 towards the front cap of the housing 11, the side member(s) 84 and thus the protrusion 83 at each side member(s) 84 catch or engage an engagement region of the housing 10. In one embodiment, the engagement region is arranged on a side guide 12 which is either mounted to the housing 10 or forms an integral part of it. Engagement of the protrusion 83 of the side (resilient) member 84 fixes the position of the cutter holder 8 when it is cocked, i.e. the cutter holder 8 is moved towards the first end of the housing carrying the front cap 11 while the tensible member 5 is positioned between first end of the housing and cutter holder 8 (see for example FIGS. 12 and 13).

An embodiment of a side guide comprising an engaging region which can be used as fixture for the cutter holder 8 is illustrated in FIG. 12. In general the side guide can have any shape as long as it provides an engagement region for the side member 84 of the cutter holder 8. In one embodiment, a side guide 12 has a rectangular shape with two stabilizing extensions 19 at each end of the side guide 12. The stabilizing extensions 19 may have a rectangular shape and being dimensioned to stabilize the position of the cutter holder 8 and the support rod holder 7 when attached to the housing 10 or to stabilize the position of the guiding member 9, if present, in the housing 10. The side guide may comprise orifices 121 for fixing the side guide 12 to the housing 10 using fixation means described herein. As shown for example in FIGS. 10, 12 and 13, the side guide can be connected to the housing 10 via a connecting beam 2001 located at the side of the housing which comprises orifices 2002 congruent in their orientation to the orifices 121 of the side guide 12. In another embodiment the side guide 12 is connected directly to the housing 12 without using a connecting beam 2001.

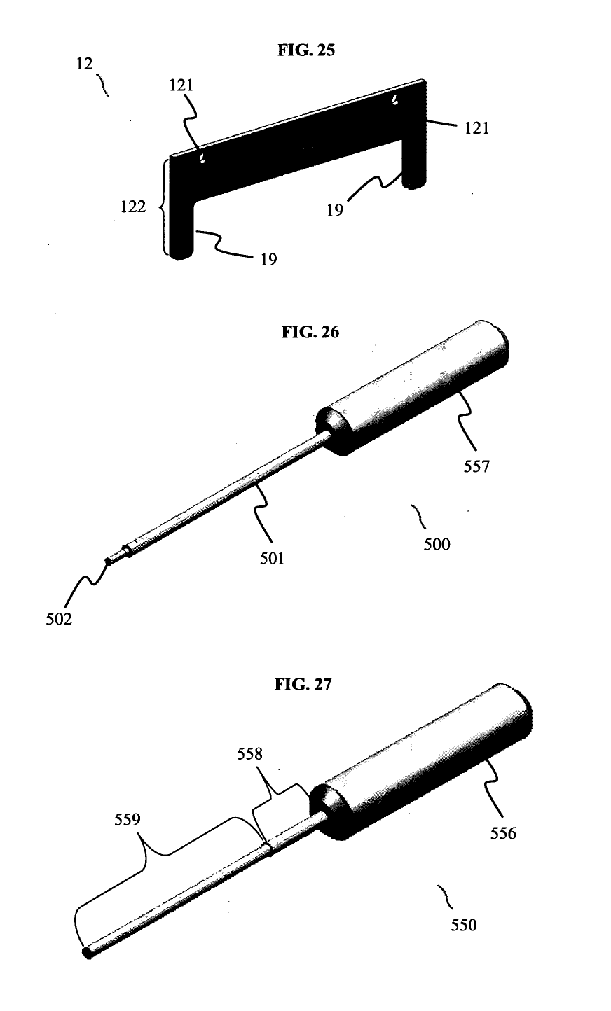

In another embodiment, the side guide 12 shown in FIG. 25 may have the shape of a rectangular thin plate mounted to either one or both sides of the housing 10 without using stabilizing extensions 19. The rigidity of the side guide 12 is adapted to allow movement of the side guide towards the cutter holder to exert a force on the side member 84 of the cutter holder 8 to release the cocked tensible member 5 attached to the cutter holder 8. In one embodiment the rigidity of the side guide 12 may be adapted only in the section comprising the engagement region 122. In an alternative embodiment in which the side guide 12 is connected to the housing 10 via a beam 2001, the beam may be made of a flexible material while the side guide 12 is of a rigid or stiff material. An exertion of force on the side guide 12 will then result in a movement of the side guide 12 due to the compression of the flexible beam 2001 rather than a deformation of the side guide 12.

Instead of using a side guide 12, the housing 10 can be shaped to comprise a flexible side portion comprising an engagement region for temporarily fixing the position of the cutter holder 8 when in its cocked position before release.

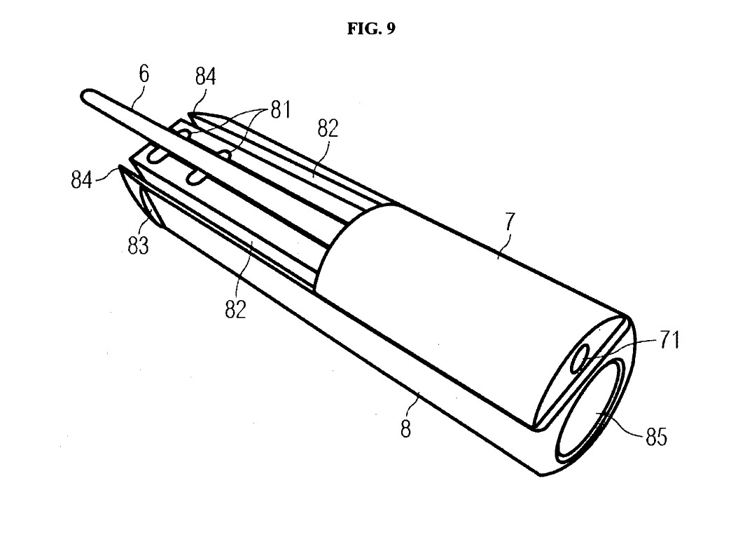

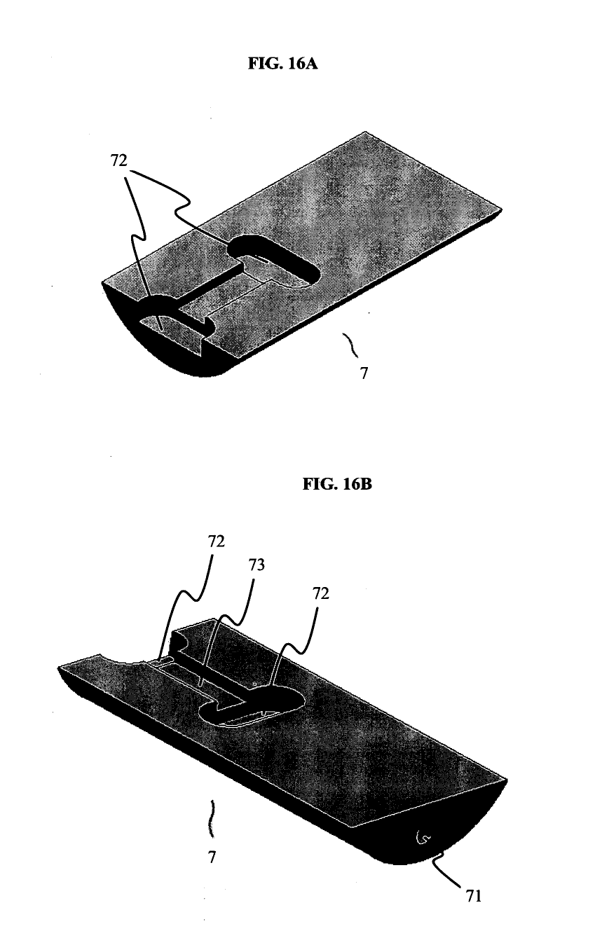

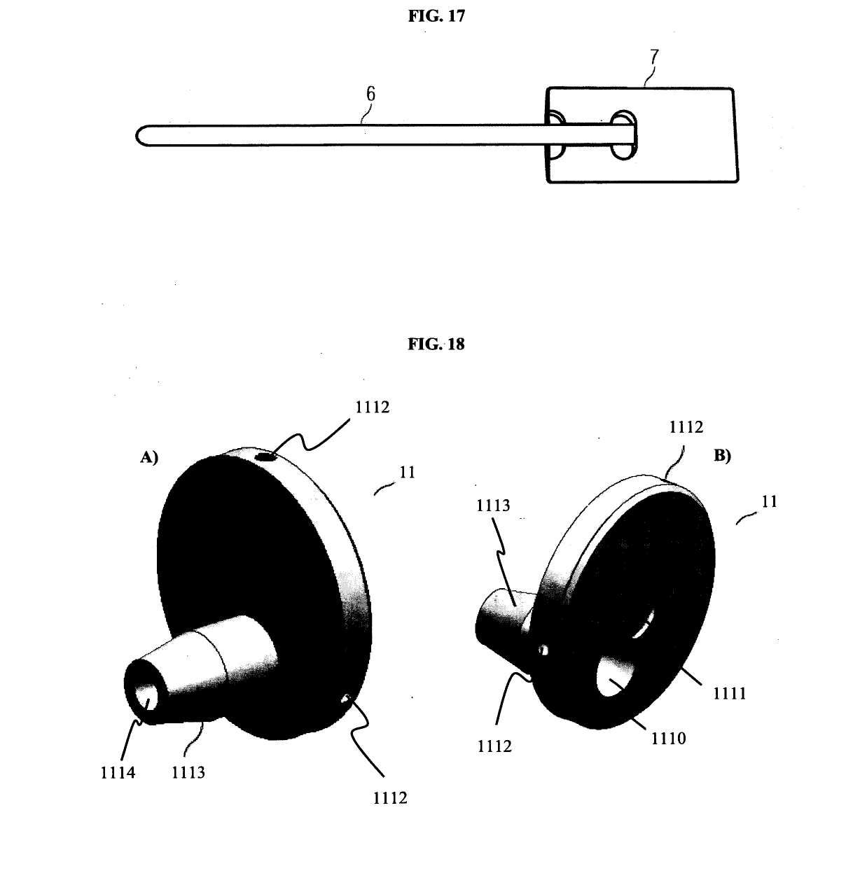

The support rod holder 7 is illustrated for example in FIGS. 9, 16 and 17. Like the cutter holder 8, the support rod holder 7 comprises a mounting point for the support rod 6. The support rod holder 7 comprises recesses 72 which are adapted to take up the support rod 6. In one embodiment, the mounting point can have the shape of a dumbbell, i.e. two rectangular shaped recesses connected by a middle section 73. The rectangular shaped recesses 72 are sunken deeper into the surface of the support rod holder 7 than the connecting middle section 73. The connecting middle section 73 can have a U-shaped profile as illustrated for example in FIG. 16B. Due to the use of two recesses connected by another higher elevated recess (middle section 73), an abutment is formed for fixing the support rod 6.

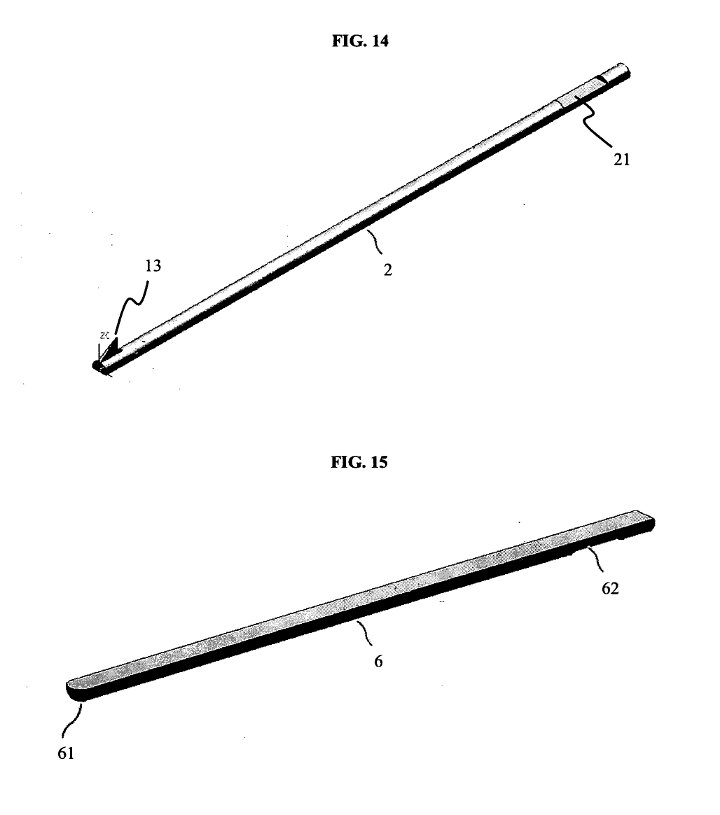

As illustrated for example in FIG. 15, the support rod 6 having a first end 61 comprises a groove 62 at the opposite end. The length and depth of the groove 62 is complementary to the length and depth of the connecting middle recess 73 of the support rod holder 7. Thus, when placed in the recess of the support rod holder 7, the support rod 6 is flush with the surface of the support rod holder 7. This connection further allows that the support rod 6 moves whenever the support rod holder 7 is moved within the housing 10. A similar structure can be found for the cutting rod 2 which is attached to the cutter holder 8. As illustrated in FIG. 22A, the cutter holder 8 can also comprise recesses 81 connected by a middle section 87. A groove 21 at the end of the cutting rod 2 (see for example FIG. 14) allows attaching the cutting rod 2 to the cutter holder 8 in the same manner as the support rod 6.

As alternative, any other method for fixing those components together can be used. For example, for permanently fixing it is also possible to weld the cutting rod 2 and the support rod 6 to the cutter holder 8 and the support rod holder 7, respectively. The support rod 6 and the cutting rod 2 can be attached to the cutter holder 8 and the support rod holder 7 in the same manner or by different means, respectively.

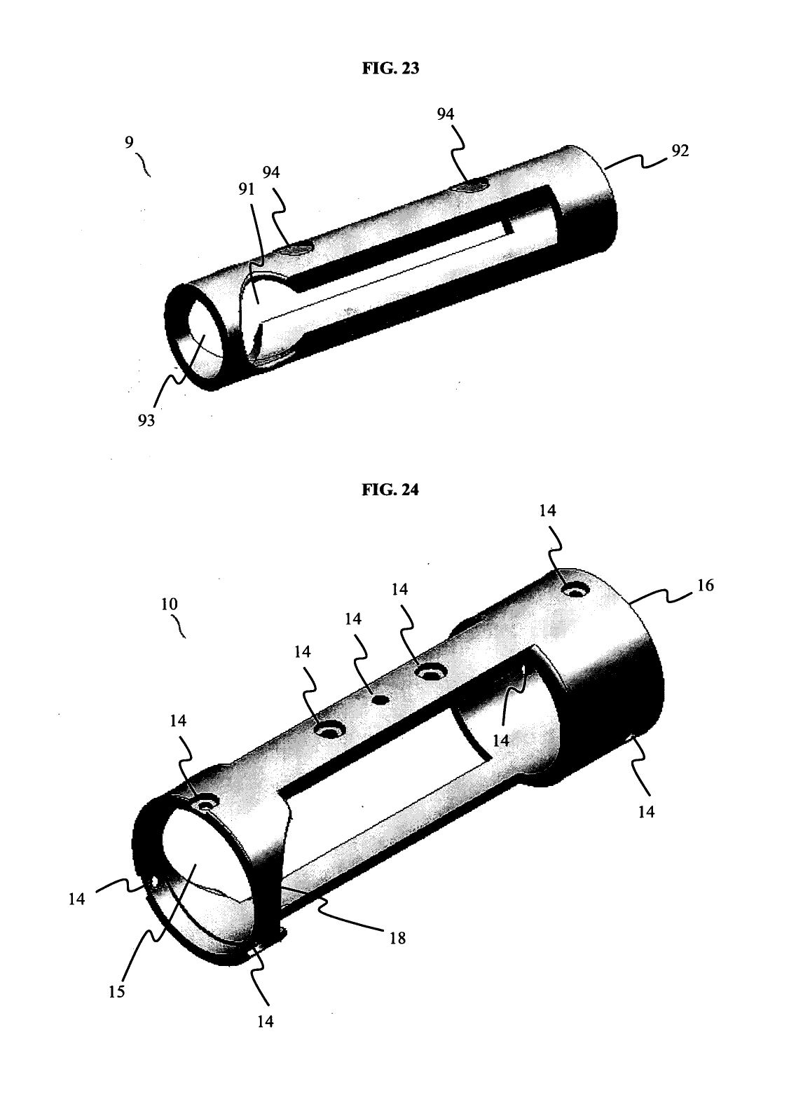

FIG. 23 shows a schematic view of a guiding member 9 which may be comprised in the housing 10. As illustrated in FIGS. 10, 12 and 13, a guiding member 9 may support the cutter holder 8 and the support rod holder 7 and can serve as a track or guard rail for the cutter holder 8 and the support rod holder 7. The cutting device 100 can also be designed without a guiding member 9 in case the housing is formed to take over the part of the guiding member 9 illustrated in the embodiment shown in the above Figures. Thus, depending on the design of the housing the use of the guiding member is optional.

In the embodiment of the guiding member 9 shown in FIG. 23, the guiding member 9 comprises a front end with a first opening 93 and a back end with a second opening 92. The guiding member 9 can have a round shape. The guiding member 9 may further comprise recesses 94 or mounting points for screws, pins, etc. (not shown) for fixing the position of the guiding member 9 within the housing 10. The recesses 94 can be positioned at the side of the guiding member 9 facing the inner side of the housing 10 within which the guiding member 9 is positioned. In another embodiment, the guiding member 9 does not comprise recesses 94. The guide member 9 is being located between the front cap 11, end cap 4 and the side guide 12. Slight vertical movement of the guide member 9 within the housing 10 is allowed and does not negatively effect the operation of the device and thus recesses 94 are not necessary.

The guiding member is in the form of a hollow tube with two strut members connecting a round front section forming the opening 93 with another round back section which forms the opening 92. The two strut members are formed like an arc to be corresponding with the curvature of the round front section and the round back section of the guiding member 9 which the two strut members connect.

In addition, a round opening 91 can be formed on one side or on both sides of the guiding member 9. The round opening 91 is adapted to give space to the thumb and index finger which are used to activate the cutting mechanism. The round opening 91 is a cut-out formed on the front side by the round front section of the housing which forms the opening 93, and the two strut members which connect the round front section and the round back section of the guiding member 9. The position of the round opening(s) 91 is corresponding to the position of the cutter holder 8 when in its cocked position. Thus, in the cocked position, the protrusion(s) of the side member(s) 84 engaging the engagement region 122 of the side guide 12 come to rest in a position which is corresponding to the position of the round opening(s) 91 in the guide member 9. The opening(s) 91 provides the necessary space which allows to exert a force onto the side member 84 which moves towards the central core region 80 of the cutter holder 8 which then results in the release of the cocked cutter holder 8.

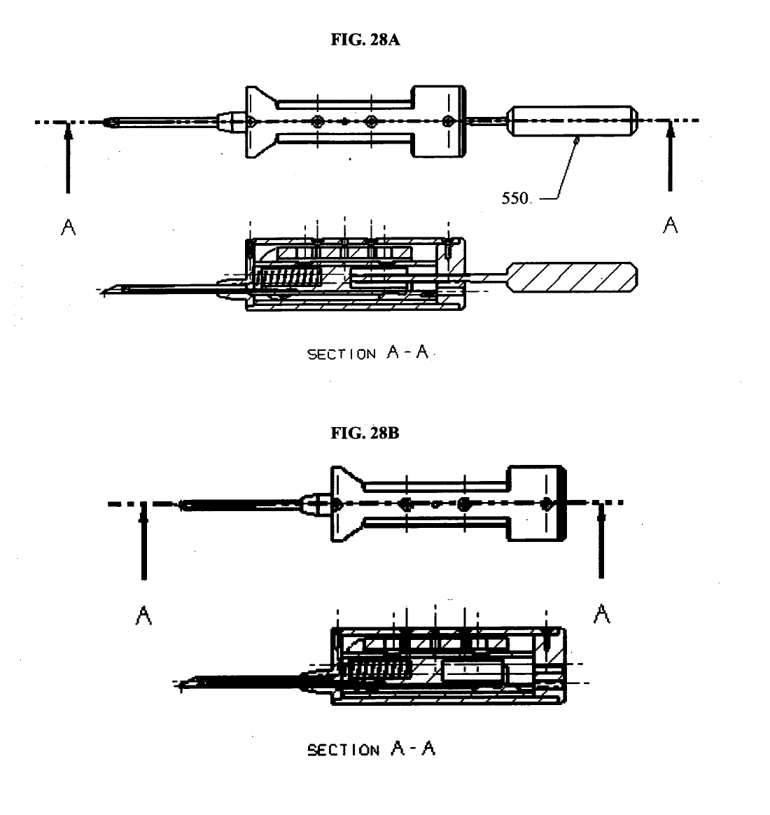

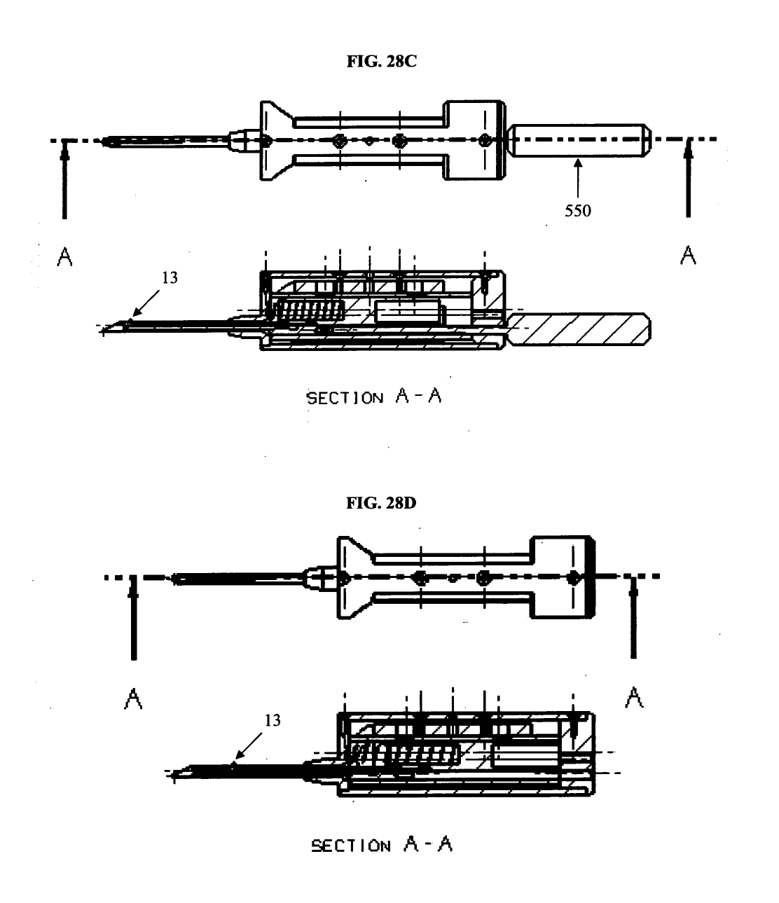

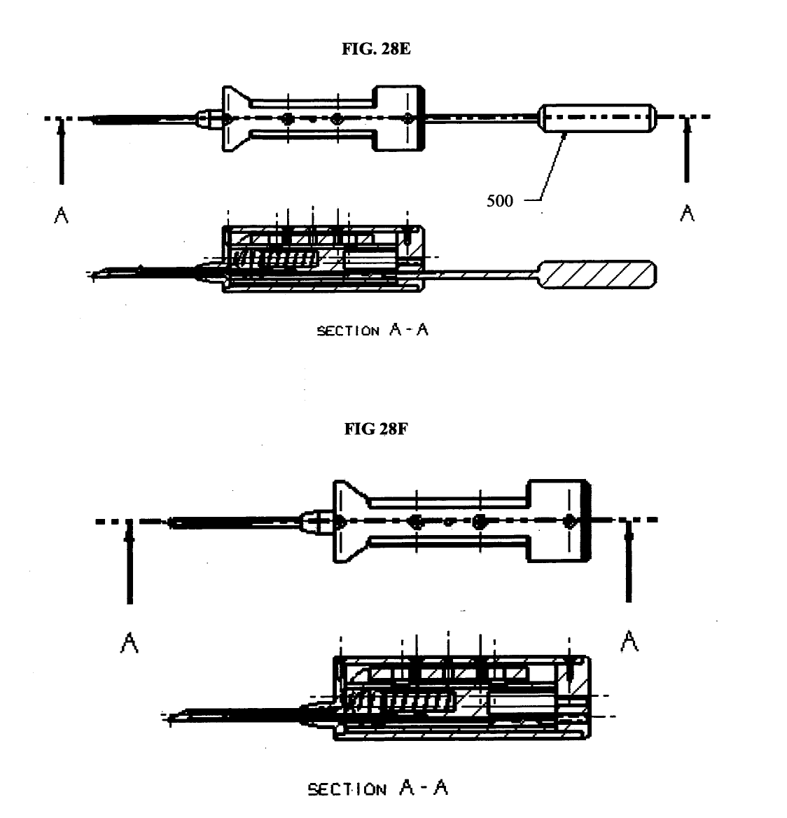

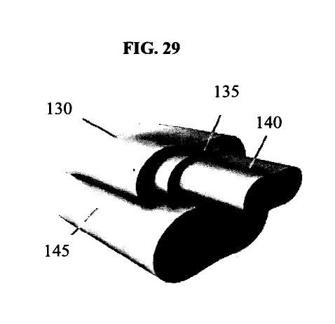

If present in the cutting device 100, the diameter of the guiding member 9 is smaller than the diameter of the housing 10 to allow placement of the guiding member 9 within the housing 10. The components of a cutting device 100, such as the guiding member 9, the housing 10 and the cutter holder 8 are designed to define in their assembled configuration a rectangular space located between the front section of the housing 10 and the end section of the housing as illustrated for example in FIGS. 7 and 13. This rectangular space defined by those components serves to mount the side guide 12 (see FIG. 7) which is used to locate and guide the guiding member 9. As can be seen in a top view of a cutting device shown for example in FIG. 28A, this results in a narrower rectangular middle section of the cutting device 100 connecting two broader end sections. This design ensures easily handling of the device by a practitioner.

In this regard, the housing 10 shown in FIG. 24 also may have a round shape but can also be of any other shape which can be easily handled by a practitioner. For example, the shape of the housing can be more ergonomic to facilitate the practitioners grip on it during use.

The housing comprises mounting points 14 for attaching the components of the housing to the frame of the housing 10 using screws or rivets or pins, to name only a few. The mounting points 14 for the front cap 11 are located in the round front section of the housing 10 and their position is corresponding to the position of the orifices 1112 at the outer perimeter of the front cap 11 which fits into the front opening of the housing 10. The mounting points 14 for the end cap 4 are located in the round end section of the housing 10 and their position is corresponding to the position of the orifices 43 at the outer perimeter of the end cap 4 which fits into the back opening of the housing 10.

The housing 10 is formed in almost the same manner as the guiding member 9 which can in one embodiment resemble the form of the housing 10. The housing may have the shape of round tube with a front section formed by an annular ring which forms a first opening 15. This first opening is for insertion of the front cap 11 which is connected to the sleeve 3. The back section of the housing 10 may also have the shape of an annular ring wherein the annular ring may be more expanded along the horizontal axis than the front section. The annular back section receives the end cap 4. The annular back section forms a second opening 16 which forms the back end of the housing 10.

Like for the guiding member 9, the round front section and back section of the housing 10 are connected by strut members. These strut members comprise the mounting points 14 for fixing the position of the guiding member 9 within the housing 10 if present. As indicated by the designation as ‘struts’, the two struts do not encase the interior of the housing but leave an open space at two sides of the housing 10. It is possible that only one strut is formed which leaves only one opening at one side of the housing. This embodiment would be useful in case the release mechanism for the cutter holder 8 comprises only one side member 84 which needs to be moved towards the central core region 80 of the cutter holder 8 to release the cocked spring 5.

The width of the two struts is also adapted to allow mounting of one or two of the connectors 2001 which can be used for mounting the side guide 12 to the housing. The round front section of the housing comprises a semicircular indentation 18 at the side of the front section facing the side which is opposite to the side to which the front cap 11 is attached. The semicircular indentation 18 can be formed at one side of the housing or on both sides. Like the round opening area 91 at the guiding member 9, the semicircular indentation is formed to provide space for direct access to the side member 84 which is necessary to operate the release mechanism for the cocked cutter holder 8 described above.

Other than to allow the direct application of force onto the side member 84 by pressing with a thumb and/or the index finger on it, the space should be sufficient to provide a firm grip for the practitioner so that the thumb and/or index finger does not slip while pressing which might change the position of the cutting blade 13 and thus the correct position at the affected area in the body of the patient.

FIG. 9 illustrates the relative position, size and shapes of the cutter holder 8 and the support rod holder 7 relative to each other in one embodiment of the present invention. In general, the cutter holder 8 and the support rod holder 7 should complement each other to form a shape which in the assembled configuration easily fits into the housing 10 or the guiding member 9. Furthermore, the cutter holder 8 holding the cutting rod 2 (not shown in FIG. 9) and the support rod holder 7 holding the support rod 6 need to be arranged relative to each other so that the cutter rod 2 and the support rod 6 are positioned in direct proximity to each other to form the complementary shape which fits into the sleeve 3 of the cutting device 100.

In one embodiment, the support rod holder has a semicircular shape with the support rod 6 mounted at the flat surface of the semicircular support rod holder 7 which is facing the flat surface of the also semicircular cutter holder 8. Thus, when placed together in the housing 10 or the guiding member 9 the cutter holder 8 and the support rod holder 7 form a circular shaped assembly. A configuration of such an assembly with a semicircular cutter rod 2 and a semicircular support rod 6 is illustrated for example in FIGS. 11 and 12 (support rod holder 7 not visible in FIGS. 11 and 12).

The cutter holder 8 and the support rod holder 7 do not necessarily have to form a circular shape together. For operation of the cutting device 100 it is sufficient when the shape of the cutter holder 8 and the support rod holder 7 are adapted so that that cutter rod 2 and the support rod 6 are in proximity to each other so that they can be easily moved in and out of the sleeve 3 together or separately from each other.

FIG. 9 also illustrates the sizes and relative positions of the cutter holder 8 and the support rod holder 7 to each other in one embodiment of the present invention. The cutter holder 8 and the support rod holder 7 are movable independently to each other along a horizontal axis within the housing 10 or guiding member 9 of the cutting device 100. The position of the support rod holder 7 depends on whether the support rod 6 is inserted into the sleeve 3 or not. In case the support rod 6 is to be introduced into the sleeve 3, the support rod holder 7 is moved towards the front of the housing 10. Referring to FIG. 9 this would mean that the support rod holder 7 is moved in the direction of the protrusion 83 which can be seen in FIG. 9. This movement would move the support rod 6 shown in FIG. 9 through the opening 1110 of the front cap 11 of the housing 10 into the sleeve 3. A position of the support rod holder 7 in which the support rod 6 attached to it is inserted into the sleeve is necessary in case the practitioner intends to raise the cutting blade 13 mounted at the front end of the cutter rod 2 to carry out a cut in the overlying tissue or tissue layer. As shown in FIG. 9, the support rod holder 7 can be moved freely along the bottom surface of the cutter holder 8. In the fully assembled cutting device 100 (see for example FIG. 7), the cutter rod 2 is normally at least partially inserted in the sleeve while the support rod is only introduced into the sleeve in case the practitioner wishes to raise the cutter rod 2 so that the cutting blade 13 protrudes through the slit 31 of the sleeve 3 for cutting tissue or a tissue layer.

In another aspect the present invention refers to a control rod 550 and a retrieval rod 500. The control rod 550 may have two functions. The first function may be to move the cutter holder 8 towards the front cap 11 of the housing 10 of the cutting device 100 until the protrusion 83 on the side member 84 of the cutter holder 3 engages the engagement region 122 of the side guide 12. The second one being moving the support rod holder 7 towards the front cap 11 of the housing 10 of the cutting device 100 to insert the support rod 6 into the sleeve 3 connected to the front cap 11 of the housing 10.

Therefore, the control rod 550 has to be adapted to be engageable with the cutter holder 8 as well as with the support rod holder 7. To engage the cutter holder 8 the control rod 550 is inserted through an opening in the end cap 4 of the cutting device 100. The end cap comprises two openings, a first opening 42 and a second opening 41. To move the cutter holder 8 the control rod 550 is inserted through the first opening 42 into the cutting device 100. The position of the first opening 42 in the end cap is aligned with the position of the cutter holder 8 in the housing 10. The control rod 550 either contacts the cutter holder 8 at its back end which faces the end cap 4 or in alternative shown in FIG. 22A is inserted through an opening 85 at the back end of the cutter holder 8 into the cutter holder 8 to contact a point inside the cutter holder 8. The round or rectangular opening 85 at the back side of the cutter holder 8 can have a cone like shape, i.e. the diameter narrows until it ends into a fixed point inside the cutter holder 8 onto which the control rod 550 comes to rest. In one embodiment, the contact point of the control rod is formed like the contact area 1111 for the spring 5 in the front cap 11 of the cutting device 100.

To avoid introducing the control rod 550 to deep into the housing and thus risk overcompression of the tensible member, the control rod comprises two different sections 559 and 558 having different diameters. The first section of the control rod 559 located at its front end has a diameter which is adapted to fit through the first opening 42 of the end cap 4. However, the second section 558 has a diameter which exceeds the diameter of the first opening 42 of the end cap 4. Thus, the control rod 550 can only be inserted into the housing 10 through the first opening 42 to the end of the first section 559.

The diameter of the second section 558 of the control rod 550 is however adapted to fit through the second opening 41 of the end cap 4. The position of the second opening 41 is aligned with the position of the support rod holder 7 in the housing 10. Thus, when inserting the control rod 550 through the second opening 41 the control rod 550 can engage the back end of the support rod holder 7, which faces the end cap 4 of the housing. Upon contact of the front end of the control rod 550 with the support rod holder 7, the support rod 6 can be moved towards the front cap 11 of the housing 10. Thus, the support rod 6 attached to the support rod holder 11 is moved into the sleeve 3. As shown in FIG. 9, the distance between the front end of the support holder 7 and the front cap 11 is much larger than the distance between the longer cutter holder 8 and the front cap 11. Therefore, the control rod 550 is inserted much deeper into the housing 10 to move the support rod holder 7 all the way to the front of the housing 10 near the front cap 11 and the sleeve 3. This is also illustrated in FIG. 28A and 28C in which it can be seen that the control rod 550 is inserted until the end of the second section 558 into the housing 10 (see FIG. 28C) when inserted into the housing 10 through the second opening 41. In comparison in FIG. 28A the control rod 550 is inserted only until the end of the first section 559 into the housing 10.

The control rod 550 moves the cutter holder 8 into the position near the front cap 11. This movement also cocks the spring and thus activates the automatic release mechanism of the device used for carrying out the cut of the tissue with the cutting blade 13. Thus, upon release of the cocked spring the cutter holder 8 is moving automatically back into the starting position by force of the relaxing spring 5. In contrast, the control rod 550 can move the support rod holder 7 into the position near the front cap 11. Since the support rod holder 7 is withdrawn manually and not via an automatic mechanism like the cutter holder 8 the retrieval rod 500 is used in one embodiment of the present invention. The retrieval rod is used to withdraw the support rod holder 7 into its starting position near the end cap 4 of the cutting device (see for example FIG. 9).

Therefore, the retrieval rod 500 comprises a front section 502 which is adapted to engage a section of the support rod holder 7 which is positioned on the opposite end of the support rod holder 7 at which the support rod 6 is mounted. In one embodiment, the front section 502 of the retrieval rod 500 comprises a thread which allows screwing the retrieval rod 500 into an opening 71 of the support rod holder 7 (see for example, FIGS. 9 and 16B). After connecting the retrieval rod to the support rod holder 7, the support rod holder 7 can be moved in its starting position by moving the retrieval rod 500 backwards through the opening 41 of the end cap 4. The diameter of the front section 502 of the retrieval rod is adapted to fit into the opening 71 of the support rod holder. The retrieval rod 500 comprises a connecting section 501. The diameter of the connecting section is adapted to fit through the opening 41 of the end cap but not through the opening 42 of the end cap. Selection of different diameters for the openings 41 and 42 of the end cap 4 and different diameters used for the control rod 550 and the retrieval rod 500 avoid that the practitioner mistakenly chooses the wrong opening for insertion of either the control rod 550 or the retrieval rod 500.

The control rod 550 and/or the retrieval rod 500 may further comprise a handle 556 and 557, respectively, which can be attached to the end of the control rod 550 or the retrieval rod 500 for ease of handling.

In another aspect, the present invention refers to a system comprising a cutter device described herein as well as a control rod and a retrieval rod described herein. Furthermore, such a system can also comprise an ultrasound device, such as a high-frequency ultrasound device operating at 17 MHz for checking the position of the sleeve of the cutting device into the affected are of the body. Sonographically assisted techniques for determining the position of a device within the human body are known in the art. For example, Jou, I. M. and Chern, T. C. (2006, Journal of Hand Surgery (British and European Volume), vol.31B, no.2, pp.191) describe a sonographically assisted percutaneous technique for releasing trigger digits which provides direct visualization.

FIG. 28 describes an exemplary embodiment illustrating the use of the different components of a system referred to herein for the operation of a cutting device described herein. At first the control rod 550 is inserted into the cutting device to cock the spring 5 and bring the cutter holder 8 and the cutter rod 2 in its forward position (FIG. 28A). After cocking of the device the cutting blade 13 of the cutter rod 2 is positioned at the beginning of the sleeve 3 at the tip 1 of the sleeve. However, the blade 13 is not yet visible because it is still hidden within the sleeve 3 (no support rod inserted). After cocking of the spring the control rod 550 is withdrawn and the cutting device 100 is now ready for insertion at the affected area of the body (FIG. 28B).

After positioning the cutting device 100 at the correct position at the affected area of the body, the cutting blade 13 needs to protrude through the slit 31 of the sleeve 3 to be able to cut through the overlying tissue. Therefore, the control rod is fully inserted through the opening 41 of the end cap 4 into the cutting device 100. Insertion moves the support rod holder 7 towards the front end of the cutting device and thus the support rod 6 into the sleeve 3. Insertion of the control rod 6 into the sleeve 3 raises the cutting blade 13 which then pierces through the overlying tissue or tissue layer (see FIG. 28C). Afterwards the control rod is removed.

To cut the desired length of the overlying tissue or tissue layer the. cutting blade 13 now needs to be moved backwards. This is achieved in the present embodiment by releasing the spring. Releasing the spring by pressing on the side member 84 of the cutter holder 8 moves the cutter holder 8 towards the end cap 4 of the cutting device. This movement moves the cutter rod 2 and the cutting blade 13 mounted on it backwards and results in cutting the overlying tissue or tissue layer (see FIG. 28D).

After the cut has been carried out, the cutting device needs to be removed. However, before removing the cutting device the cutting blade 13 is lowered back into the sleeve to avoid damaging the surrounding tissue when removing the cutting device from the body. Therefore, the retrieval rod 500 is inserted into the device 100 through the opening 41 in the end cap of the cutting device 100. The retrieval rod 500 engages the support rod holder 7 through the opening 71. Moving the support rod holder 7 backwards removes the support rod 6 out of the sleeve 3 (see FIG. 28E). Now the cutting rod 2 with the cutting blade 13 sinks back into its lower position within the sleeve 3 in which the cutting blade 13 does not protrude through the slit 31 (see FIG. 28F). Afterwards the cutting device can be safely removed without damaging the surrounding tissue.

The same operation can also be carried out with the cutting device illustrated in FIG. 1.

In further embodiments, the shape of the sleeve can be curved which would allow to reach positions within the body which cannot easily be reached with a straight sleeve. In this case the support rod 6 and the cutter rod 2 can be made of flexible materials which allow their movement around bends or curves in the sleeve.

The cutting device and its components can be made of any material which is used for apparatuses in clinical applications. For example, the devices described herein can be made of materials which are easily sterilized, such as stainless steel, titanium and plastic.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### LIST OF REFERENCE SIGNS

1 Tip of sleeve
2 Cutting rod
3 Sleeve
4 End cap of housing
5 Spring
6 Support rod
7 Support rod holder
8 Cutter holder
9 Guiding member
10 Housing
11 Front cap of housing
12 Side guide
13 Cutting blade
14 Mounting point
15 First opening of housing
16 Second opening of housing
17 Connecting collar
18 Semicircular indentation in front section of the housing
19 Stabilizing extension
21 Groove for attaching cutting rod to cutter holder
31 Slit
32 Opening at first end of sleeve
41 Second opening in end cap
42 First opening in end cap
43 Opening for fixation means
61 Front end of support rod
62 Groove for attaching support rod to support rod holder
71 Opening for retrieval rod
72 Recess for attachment to support rod
73 Connecting recess
80 Central core region
81 Recess for holding cutting rod
82 Slit in cutter holder
83 Protrusion for engaging side guide
84 Side member
85 Opening in cutter holder for inserting control rod
86 Opening for spring
87 Connecting recess
91 Rounded opening area for side guide
92 Opening of guiding member towards end cap
93 Opening of guiding member towards front cap
94 Contact point for fixation means
121 Opening for fixation means
122 Engagement region of side guide
130 Pulley
135 Tenosynovium
140 Tendon
145 Bone
500 Retrieval rod
501 Connecting section between handle and tip of retrieval rod
502 Front section of retrieval rod
550 Control rod
556 Handle
557 Handle of retrieval rod
558 Second section of control rod
559 First section of control rod
1110 Opening in front cap for support rod and cutting blade
1111 Contact area for spring
1112 Opening for fixation means
1113 Connecting member
1114 Opening for inserting sleeve
2001 Beam for connecting side guide
2002 Hole for inserting fixation means
3000 Cutting rod
3001 Cutting blade
3002 Tip of sleeve
3003 Support rod guiding member
3004 Positioning member
3005 Handle of support rod guiding member
3100 Sleeve
3110 Slit in sleeve
3201 Support rod

1. A cutting device for cutting tissue comprising:
a sleeve having a first end and a second end, the sleeve comprises a slit extending from a position at the first end of the sleeve and wherein the first end of the sleeve is shaped to allow insertion of the sleeve between two tissue layers;
a cutting rod carrying a cutting blade at a first end, the cutting rod being insertable into the sleeve through the second end of the sleeve and being movable within the sleeve; wherein the cutting rod is positioned within the sleeve so that the cutting blade can protrude through the slit of the sleeve; and
a support rod, the support rod being insertable into the sleeve through the second end of the sleeve and being movable within the sleeve;
wherein the cutting rod and the support rod fit together into the sleeve; and wherein when the cutting rod and the support rod are arranged together within the sleeve the cutting blade of the cutting rod is protruding through the slit of the sleeve.

2. The cutting device of claim 1, wherein the cutting rod and the support rod are independently of each other movable within the sleeve and insertable in the sleeve.

3. The cutting device according to claim 1 or 2, wherein the cutting rod and the support rod fill out the entire space of the sleeve when fully inserted into the sleeve during use.

4. The cutting device of claim 3, wherein the shape of the cutting rod and the shape of the support rod is the same or different from each other.

5. The cutting device of claim 4, wherein the sleeve is round.

6. The cutting device of any one of the preceding claims, wherein the cutting rod and the support rod have a semicircular shape.

7. The cutting device of any one of the preceding claims, wherein the first end of the sleeve comprises a tip with a tapered shape.

8. The cutting device of claim 7, wherein the first end of the sleeve comprises a tip beveled along a vertical axis.

9. The cutting device of any one of the preceding claims, further comprising a housing comprising a first end and a second end and being connected to the sleeve via the first end;
wherein the housing comprises a movable cutter holder and a movable support rod holder;
wherein the cutter holder supports the cutting rod and the support rod holder supports the support rod;
wherein the cutter holder and the support rod holder are movable independently from each other during use; and
wherein a movement of the cutter holder within the housing allows moving the cutter rod within the sleeve and a movement of the support rod holder allows moving the support rod within and out of the sleeve during use.

10. The cutting device of claim 9, wherein the first end of the first housing is closed with a front cap which is connected to the sleeve and the second end is closed with an end cap.

11. The cutting device of claim 10, wherein the cutter holder and the support rod holder are movably supported by a guiding member and wherein the guiding member is comprised in the housing.

12. The cutting device of any one of claims 9 to 11, further comprising a tensible member positioned between the first end of the first housing and the cutter holder such that a movement of the cutter holder towards the first end of the first housing cocks the tensible member and positions the cutter holder in a cocked position.

13. The cutting device of claim 12, further comprising at least one side guide mounted at a side of the housing and comprising an engaging region located near the first end of the housing; wherein a protrusion located at at least one side of the cutter holder engages the engaging region of the at least one side guide through an opening of the housing to keep the cutter holder in the cocked position when the tensible member is cocked during use.

14. The cutting device of claim 13 comprising two side guides, wherein the two side guides are positioned at opposing sides of the housing.

15. The cutting device of claim 13 or 14, wherein the cutter holder further comprises at least one slit separating the cutter holder into a central core region and a side member;
wherein the central core region supports the cutting rod and provides a contact region for the tensible member;
wherein the side member comprises the protrusion which engages the engaging region of the at least one side guide to keep the cutter holder in the cocked position when the tensible member is cocked during use; and
wherein the side member is movable towards the central core region from which it is separated by the slit upon exerting pressure on the side member; wherein a movement of the side member of the cutter holder towards the central core region allows to uncock the cutter holder from its cocked position by disengaging the connection between the protrusion and the side guide.

16. The cutting device of claim 15, wherein the cutter holder comprises two slits separating a section of the cutter holder into a central core region and two side members which are located at opposing sides of the cutter holder.

17. The cutting device of any one of claims 9 to 16, wherein the second end of the housing comprises a first opening for inserting a control rod into the device, wherein the first opening is positioned to allow the control rod to engage the cutter holder and to move the cutter holder towards the first end of the housing when inserted into the device.

18. The cutting device of any one of claims 9 to 17, wherein the second end comprises a second opening for inserting a retrieval rod into the device or for inserting a control rod into the device,
wherein the second opening is positioned to allow a retrieval rod to engage the support rod holder and to move the support rod holder towards the second end of the housing when inserted into the device and thus moving the support rod supported by the support rod holder out of the sleeve; or
wherein the second opening is positioned to allow the control rod to move the support rod holder towards the first end of the housing and thus moving the support rod supported by the support rod holder into the sleeve.

19. The cutting device of any one of claims 1 to 8, wherein the cutting device further comprises a support rod guiding member connected to the support rod and comprising a through hole extending from one end of the support rod guiding member to the other end.

20. The cutting device of claim 19, wherein the cutting rod is adapted to fit through the through hole of the support rod guiding member.

21. The cutting device of any one of claims 1 to 8 and 19 to 20, wherein the sleeve is connected to a positioning member adapted to allow positioning of the sleeve at a specific position in a tissue of a subject.

22. A control rod for use in a device of any one of claims 1 to 18, the control rod having a first end and a second end, wherein the control rod comprises a first section and a second section, wherein the first and the second section have different diameters; wherein the diameter of the first section located at the first end of the rod is smaller than the diameter of the second section following the first section.

23. The control rod of claim 22, wherein the control rod further comprises a handle located at the second end of the control rod.

24. The control rod of claim 22 or 23, wherein the diameter of the first section is adapted to be insertable through the first opening of the second end of the housing of the cutting device and the diameter of the second section is adapted to be larger than the diameter of the first opening of the second end of the housing of the cutting device.

25. The control rod of any one of claims 22 to 24, wherein the diameter of the second section is adapted to be insertable through the second opening of the second end of the housing of the cutting device.

26. A retrieval rod for use in a device of any one of claims 1 to 18, the retrieval rod having a first end and a second end, wherein the retrieval rod comprises an engaging member at the first end of the retrieval rod for engaging the support rod holder.

27. The retrieval rod of claim 26, wherein the retrieval rod further comprises a handle located at the second end of the control rod.

28. The retrieval rod of claim 26 or 27, wherein the engaging member has a thread.

29. A support rod guiding member for use in a device of any one of claims 1 and 19 to 21, the support rod comprising a through hole extending from one end of the support rod guiding member to the other end.

30. A system for cutting tissue comprising:
a cutting device of any one of claims 1 to 18;
a control rod of any one of claims 22 to 25; and
a retrieval rod of any one of claims 26 to 28.

31. The system for cutting tissue of claim 30 further comprising an ultrasound device.
